(19) United States
(12) Patent Application Publication
(10) Pub. No.: US 2012/0009271 A1
(43) Pub. Date: Jan. 12, 2012

(54) METHOD OF DISEASE-INDUCED AND RECEPTOR-MEDIATED STEM CELL NEUROPROTECTION

(75) Inventors: Cesario V. Borlongan, Tampa, FL (US); Ornella Parolini, Brescia (IT)

(73) Assignees: MEDICAL COLLEGE OF GEORGIA, Augusta, GA (US); UNIVERSITY OF SOUTH FLORIDA, Tampa, FL (US)

(21) Appl. No.: 13/230,354

(22) Filed: Sep. 12, 2011

Related U.S. Application Data
(63) Continuation of application No. PCT/US2010/027122, filed on Mar. 12, 2010.
(60) Provisional application No. 61/159,645, filed on Mar. 12, 2009.

(51) Int. Cl. A61K 35/50; A61P 9/10; A61P 25/28; A61P 25/16; C12N 5/071; C12N 5/0775

(52) U.S. Cl. 424/583; 435/375; 435/366; 435/371

(57) ABSTRACT

Stem cells are exposed to disease condition (the OGD stroke model), that mimics the target disease (stroke), allowing the stem cells to exert better neuroprotective effects. Thus, the present technology demonstrates a disease-tailored stem cell therapy. The present invention discloses that the administration of a therapeutically effective amount of amnion derived stem cells concomitantly with a therapeutically effective dose of melatonin provides additive/synergistic neuroprotective effects. Moreover, the present invention offers an equally robust technology employing a receptor-regulated mechanism, whereby stem cells can be enhanced (melatonin treatment) over their basal level (lack of melatonin treatment), facilitating a regulation of stem cells.

### CROSS REFERENCE TO RELATED APPLICATION

This application claims priority to currently pending U.S. Provisional Patent Application No. 61/159,645, entitled “Disease-induced and Receptor-mediated Stem Cell Neuroprotection”, filed on Mar. 12, 2009, the contents of which are herein incorporated by reference.

### FIELD OF INVENTION

This invention relates to stem cell therapy. Specifically this invention relates to neuroprotection through the administration of amnion derived stem cells and melatonin.

### BACKGROUND OF THE INVENTION

Cell therapy has been proposed for treatment of chronic inflammation and immune alterations (multiple sclerosis, inflammatory bowel disease (IBD), arthritis, graft versus host disease) and ischemia (coronary/peripheral artery disease, stroke). Current evidence suggests that cell replacement is likely not the major mechanism by which cell therapy confers functional benefit. Despite reports of bone marrow-derived stem cells differentiating into neural cells or “fusing” with diseased neurons, the number of engrafted cells is low and likely insufficient to account for the observed functional improvements. Rather, increasing experimental data indicate that stem or progenitor cells, such as mesenchymal stromal cells (MSC), act beneficially by exerting trophic effects on host cells, reducing apoptotic cell death and stimulating angiogenesis, neurogenesis, vasculogenesis, among other regenerative processes. Indeed, administration of MSC after cerebral ischemia leads to increased angiogenesis, neurogenesis, synaptogenesis, and oligodendrogenesis, with axonal sprouting either directly or by stimulating endogenous cells to secrete trophic and protective factors. Concomitantly, the immunomodulatory effects of MSC, independently of the source, blunt the inflamatory response and allow tissue remodeling after injury, resulting in reduced numbers of fibroblasts and less scarring in the heart, lung, and kidney and less astroglial scarring in the brain. (Parolini, O. et al. (2010) Toward Cell Therapy Using Placenta-Derived Cells: Disease Mechanisms, Cell Biology, Preclinical Studies, and Regulatory Aspects at the Round Table, STEM CELLS AND DEVELOPMENT, Vol. 19, No. 2:143-154)

Emerging evidence indicates that after ischemic stroke the peripheral immune response is activated and immune cells migrate to the brain and contribute to cerebral injury. Intravenous administration of hematopoietic stem cells and umbilical cord blood cells reduces cerebral ischemic injury and inflammation at least partly by interfering with splenic and lymphoid activation, suggesting that intravenous delivery might be preferable in pathologies involving inflammation activation and immune response. (Parolini, 2010)

Although the anti-inflammatory and immunomodulatory effects discussed above pertain to stem or progenitor cells in general, accumulating evidence suggests that similar mechanisms might also accompany placenta-derived cells. Human placenta represents a reservoir of progenitor/stem cells that can be used in cell therapy applications. Placental tissue exhibits phenotypic plasticity of many of the cell types isolated from this tissue and also contains cells which display immunomodulatory properties. Both factors are important to cell therapy-based clinical applications. (Parolini, O. et al., Isolation and Characterization of Cells from Human Term Placenta: Outcome of the First International Workshop on Placenta Derived Stem Cells, Stem Cells Express, Nov. 8, 2007 p. 1-11)

The fetal adnexa is composed of the placenta, fetal membranes and umbilical cord. The placenta is discoid in shape with a diameter of 15-20 cm and a thickness of 2-3 cm. (Parolini 2007) The placenta is a fetomaternal organ consisting of 2 components: the maternal component, termed the decidua, originating from the endometrium, and the fetal component, including the fetal membranes—amnion and chorion—as well as the chorionic plate, from which chorionic villi extend and make intimate contact with the uterine decidua during pregnancy. (Parolini 2010)

From the margins of the chorionic disc extend the fetal membranes, amnion and chorion, which enclose the fetus in the amniotic cavity, and the endometrial decidua. The chorionic plate is a multilayered structure which faces the amniotic cavity. It consists of two different structures: the amniotic membrane (composed of epithelium, compact layer, amniotic mesoderm and spongy layer) and the chorion (composed of mesenchyme and a region of extravillous proliferating trophoblast cells interposed in varying amounts of Langhans fibrinoid, either covered or not by syncytiotrophoblast). Villi originate from the chorionic plate and anchor the placenta through the trophoblast of the basal plate and maternal endometrium. From the maternal side, protrusions of the basal plate within the chorionic villi produce the placental septa, which divide the parenchyma into irregular cotyledons. (Parolini 2007)

Some villi anchor the placenta to the basal plate, while others terminate freely in the intervillous space. Chorionic villi present with different functions and structure. In the term placenta, the stem villi show an inner core of fetal vessels with a distinct muscular wall, and connective tissue consisting of fibroblasts, myofibroblasts, and dispersed tissue macrophages (Hofbauer cells). Mature intermediate villi and term villi are composed of capillary vessels and thin mesenchyme. A basement membrane separates the stromal core from an uninterrupted multinucleated layer, called syncytiotrophoblast. Between the syncytiotrophoblast and its basement membrane are single or aggregated Langhans' cytotrophoblastic cells, commonly called cytotrophoblast cells. (Parolini 2007)

Fetal membranes continue from the edge of the placenta and enclose the amniotic fluid and the fetus. The amnion is a thin, avascular membrane composed of an epithelial layer and an outer layer of connective tissue, and is contiguous, over the umbilical cord, with the fetal skin. The amniotic epithelium (AE) is an uninterrupted, single layer of flat, cuboidal and columnar epithelial cells in contact with amniotic fluid. It is attached to a distinct basal lamina that is, in turn, connected to the amniotic mesoderm (AM). In the amniotic mesoderm closest to the epithelium, an acellular compact layer is distinguishable, composed of collagens I, III and fibronectin. Deeper in the AM, a network of dispersed fibroblast-like mesenchymal cells and rare macrophages are observed. Very recently, it has been reported that the mesenchymal layer of amnion indeed contains two subfractions, one having a mesenchymal phenotype which is referred to as amniotic mesenchymal stromal cells (AMSC), and the second containing monocyte-like cells. (Parolini 2007)

A spongy layer of loosely arranged collagen fibers separates the amniotic and chorionic mesoderm. The chorionic membrane (chorion leave) consists of mesodermal (CM) and trophoblastic (CT) regions. Chorionic and amniotic mesoderm are similar in composition. A large and incomplete basal lamina separates the chorionic mesoderm from the extravillous trophoblast cells. The latter, similar to trophoblast cells present in the basal plate, are dispersed within the fibrinoid layer and express immunohistochemical markers of proliferation. The Langhans' fibrinoid layer usually increases during pregnancy and is composed of two different types: a matrix type on the inner side (more compact) and a fibrin type on the outer side (more reticulate). At the edge of the placenta and in the basal plate, the trophoblast interdigitates extensively with the deciduas. (Parolini 2007)

Neurological disorders represent a significant burden to western societies, highlighting the need to develop effective therapies. Stroke is a serious neurological disorder representing a current unmet medical condition of significance worldwide. In the United States, stroke is the third leading cause of death and the primary cause for disability. Cell replacement therapy has been proposed as a basis for new treatment strategies for a broad range of neurological diseases; however, the paucity of suitable cell types has so far hampered the development of this promising therapeutic approach. (Parolini, 2010)

Recent studies have implicated the abnormal accumulation of free radicals in neurodegenerative disorders. Free radical scavengers have been shown to protect against cell death. Melatonin, the main secretory product of the pineal gland, is well known for its functional interactions with the neuroendocrine axis and with circadian rhythms. Melatonin, (N-acetyl-5-methoxytryptamine), is a highly potent free radical scavenger and indirect antioxidant and has been shown to exert neuroprotection in models of brain and spinal cord trauma (U.S. Pat. No. 6,075,045, herein incorporated in its entirety by reference), cerebral ischemia, and excitotoxicity. (Borlongan, C. et al., Glial Cell Survival Is Enhanced During Melatonin-Induced Neuroprotection Against Cerebral Ischemia, The FASEB Journal (2000) 14:1307-1317)

Melatonin has been shown to exert neuroprotection in a variety of oxidative stress-associated neuropathologies, including brain and spinal cord trauma, cerebral ischemia, neurotoxicity, and models of Parkinson's and Alzheimer's diseases. It has been reported that melatonin exerts its neuroprotective action in various neurodegenerative disorders through its antioxidant and free radical scavenging property. However, the specific mechanism by which melatonin induces neuroprotection is unknown. It was previously unknown whether MelR1 or MelR2 melatonin receptors play any role in neuroprotection, or if the neuroprotection is attributable to the free radical scavenging property of melatonin. The inventors have discovered that stimulation of the MelR1 melatonin receptor by melatonin exerts a neuroprotective effect. The inventors have also discovered that the administration of melatonin with amnion epithelial cells (AECs) exerts a synergistic neuroprotective effect that is due to stimulation of the melatonin receptor 1 (MelR1).

### SUMMARY OF INVENTION

The mechanisms underlying stem cell therapeutic benefits remain poorly understood. Unraveling these mechanisms will lead to novel technologies directed at exploiting stem cells to exert a highly regulated therapeutic outcome in disease models. The inventors have discovered a disease-tailored stem cell therapy whereby stem cells are exposed to disease condition (the OGD stroke model), that mimics the target disease (stroke), allowing the stem cells to exert better neuroprotective effects. Moreover, the present invention offers an equally robust technology employing a receptor-regulated mechanism, whereby stem cells can be enhanced (melatonin treatment) over their basal level (lack of melatonin treatment), facilitating a regulation of stem cells. One can envision that in contemplating with translational and/or clinical potential of both technologies, the disease-tailored technology can be used in stem cell preparation prior to transplantation, in that stem cells are exposed to stroke model in vitro when desired for transplanting stem cells in stroke, or exposed to other particular in vitro disease models (e.g., Parkinson's disease, Alzheimer's disease, etc). In parallel, the melatonin receptor-based technology can be used for regulating stem cells after transplantation. Both technologies are deemed novel strategies designed to improve and to control the functional outcome of stem cell therapy.

One embodiment of this invention is a method of treating a patient suffering from a neurodegenerative disorder comprising administering a therapeutically effective amount of human placenta derived stem cells. These cells are preferably amnion derived epithelial cells or amnion derived mesenchymal cells. Preferably, the cells are exposed to a disease model prior to administration to the patient. The cells can also be concomitantly administered with a therapeutically effective dose of melatonin to enhance the neuroprotective effect.

The neurodegenerative disorder being treated can be stroke, Alzheimer' s Disease, Parkinson's disease and ischemia.

In another embodiment is a method of regulating stem cells comprising stimulating the MelR1 receptor with melatonin. The stem cells can be human placenta derived stem cells and are preferably amnion epithelial cells or amnion mesenchymal cells.

Another embodiment provides a method of enhancing neuroprotection through the stimulation of MelR1. The stimulation of MelR1 can be accomplished through the administration of human placental derived stem cells and melatonin. The human placental derived cells can be amnion epithelial cells or amnion mesenchymal cells.

### BRIEF DESCRIPTION OF THE DRAWINGS

For a fuller understanding of the invention, reference should be made to the following detailed description, taken in connection with the accompanying drawings, in which:

FIG. 1 is a graph showing OGD-exposed human amnion stem cells exert enhanced neuroprotection. In vitro experimental stroke (oxygen glucose deprivation, OGD) significantly increases levels of neurotrophic factors secreted by cultured human amnion stem cells compared to control, standard medium

FIG. 2 is a graph showing that both OGD-exposed human amnion stem cells and the conditioned media from OGD-exposed amnion cells exert enhanced neuroprotection. In vitro experimental stroke (oxygen glucose deprivation, OGD) significantly increases levels of neurotrophic factors secreted by cultured human amnion stem cells compared to control, standard medium.

FIG. 3 is a chart showing the results of an ELISA analysis illustrating that neurotrophic factors VEGF and GDNF are increased in OGD-exposed amnion cells.

FIGS. 4A-F are a series of images showing expression of Melatonin R1 (MelR1) in Cultured Human Amnion Stem Cells (scale bars; 40 μm). (A) Expression of Human Specific Nuclear Antigen (HuNu) on day 3; (B) Expression of Melatonin R1 receptor on day 3; (C) Merged image of HuNu and Melatonin 1 receptor expression on day 3; (D) Expression of Human Specific Nuclear Antigen (HuNu) on day 5; (B) Expression of Melatonin R1 receptor on day 5; (C) Merged image of HuNu and Melatonin R1 receptor expression on day 5.

FIGS. 5A-C are a series of images showing a lack of Melatonin R2 (MelR2) Expression in Cultured AECs (scale bars; 40 μm). (A) Expression of Human Specific Nuclear Antigen (HuNu) on day 5; (B) Expression of Melatonin R2 receptor on day 5; (C) Merged image of HuNu and Melatonin R2 receptor expression on day 5.

FIGS. 6A-D are a series of images showing the neuroprotective effect of Melatonin (100 μM) against oxidative stress (H202). (A) Control followed by H202; (B) Treatment with melatonin followed by H202; (C) Tryptan blue stained control followed by H202; (D) Tryptan blue stained melatonin treated cells followed by H202.

FIGS. 7A-D are a series of images showing the anti-oxidant effect of Melatonin against oxidative stress (H202, 100 μM ; Melatonin, 100 μM). (A) Control; (B) Control followed by oxidative stress; (C) Melatonin treated cells followed by oxidative stress; (D) Melatonin treated cells.

FIGS. 8A and B are a series of graphs showing the anti-oxidant effect of Melatonin (H202, 100 μM; Melatonin, 100 μM). (A) Pre-treatment of cells with melatonin protects against cell death in Hoechest 33258 labeled cells; (B) Pretreatment of cells with melatonin protects against cell death as measured by tryptan blue staining.

FIGS. 9A-D are a series of images showing the differentiation of cultured human amnion stem cells after administering 100 μM Melatonin. (A) expression of TuJ1; (B) expression of GFAP; (C) Hoechst stained cells; (D) merged image showing expression of TuJ1 and GFAP.

FIGS. 10A-F are a series of images showing differentiation of cultured human amnion stem cells after administering 100 μM melatonin. (A) expression of TuJ1; (B) Hoechst cells; (C) merged image showing expression of TuJ1 in Hoechst cells; (D) expression of GFAP; (E) Hoechst stained cells; (F) merged image showing expression of GFAP in Hoechst stained cells.

FIGS. 11A-D are a series of images showing differentiation of cultured human amnion stem cells after administering 100 μM melatonin. (A) expression of Hu Nestin; (B) expression of MT1; (C) Hoechst stained cells; (D) merged image showing expression of Hu Nestin and MT1.

FIGS. 12A-F are a series of images showing differentiation of cultured human amnion stem cells in standard medium (Control). (A) expression of TuJ1; (B) Hoechst stained cells; (C) merged image showing TuJ1 expression in Hoechst stained cells; (D) expression of GFAP; (E) Hoechst stained cells; (F) merged image showing GFAP expression in Hoechst stained cells.

FIGS. 13A-D are a series of images showing differentiation of cultured human amnion stem cells in standard medium (Control). (A) expression of Hu Nestin; (B) expression of MT1; (C) Hoechst stained cells; (D) merged image showing expression of Hu Nestin and MT1 in Hoechst stained cells.

FIGS. 14A and 14B are a series of graphs showing that melatonin enhanced human amnion stem cell differentiation into neuronal cells as revealed by neuronal phenotype expression and neuron-like morphology. (A) total TuJ1 positive cells in 5 fields for neuronal cells (dendrite +/−) for control and melatonin treated cells; (B) total positive cells in 5 fields for nestin and nestin/MelR1 positive cells for control and melatonin treated cells.

FIG. 15 is a graph illustrating receptor specific neuroprotection. Pre-treatment of amnion cells with MelR1 antibody, but not MelR2 antibody, blocks neuroprotective effects of the stem cells on primary rat cells against in vitro experimental stroke. These results further support the claim that amnion-derived stem cells afford neuroprotection specifically via MelR1 receptor.

FIG. 16 is a graph illustrating that combined treatment administering melatonin and amnion-derived stem cells enhances neuroprotection. As shown by the figure, combined treatment with melatonin and amnion-derived stem cells enhanced the neuroprotective effects against in vitro experimental stroke. These data also lend support that stimulating the MelR1 could aid in the therapeutic benefits of amnion-derived stem cells.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

“Patient” is used to describe an animal, preferably a human, to whom treatment is administered, including prophylactic treatment with the cells and/or compositions of the present invention. For treatment of those infections, conditions or disease states which are specific for a specific animal such as a human patient, the term patient refers to that specific animal. The term “donor” is used to describe an individual (animal, including a human) who or which donates placental tissue or placental derived cells for use in a patient.

The “therapeutically effective amount” for purposes herein is thus determined by such considerations as are known in the art. A therapeutically effective amount is used to describe concentrations or amounts of components such as differentiation agents, stem cells, precursor or progenitor cells, specialized cells, such as neural and/or neuronal or glial cells, compounds such a melatonin that stimulate receptors such as the melatonin 1 receptor and/or other agents that are effective for producing an intended result including differentiating stem and/or progenitor cells into specialized cells, such as neural, neuronal and/or glial cells, or treating a neurological disorder such as Alzheimer's disease or Parkinson's disease, or other pathologic condition including damage to the central nervous system of a patient, such as a stroke, heart attack, ischemia or accident victim or for effecting a transplantation of those cells within the patient to be treated. Compositions according to the present invention may be used to affect a transplantation of the placental derived cells within the composition to produce a favorable change in the brain or spinal cord, or in the disease or condition treated, whether that change is an improvement such as stopping or reversing the degeneration of a disease or condition, reducing a neurological deficit or improving a neurological response, or a complete cure of the disease or condition treated. In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of the animal and the route of administration.

The term “stem cell” refers to a master cell that can reproduce indefinitely to form the specialized cells of tissues or organs. A stem cell can divide to produce two daughter stem cells or one daughter stem cell and one “progenitor” cell which then proliferates into the tissue's mature, fully-formed cells. As used herein, the term “stem cell” includes multipotent and pluripotent stem cells.

The term “pluripotent cell” refers to a cell that has complete differentiation versatility, i.e. the capacity to grow into any of the mammalian body's cell types, except for the extraembryonic tissues. A pluripotent stem cell can be self-renewing and can remain dormant or quiescent within a tissue.

The term “multipotent stem cell” refers to a cell that has the capacity to grow into two or more different cell types within a given tissue or organ. A multipotent stem cell may have the capacity to be pluripotent.

The term “progenitor cell” refers to a cell that is committed to differentiate into a specific cell type or form a specific type of tissue.

The term “placenta derived stem cells” is used herein to refer to a cell that is derived from the placenta. The placental derived stem cells can be administered systemically or to a target anatomical site, permitting the cells to differentiate in response to the physiological signals encountered by the cell (e.g., site-specific differentiation).

Alternatively, the cells may undergo ex vivo differentiation prior to administration into a patient. Placenta-derived stem cells are further divided into human amniotic epithelial cells (hAEC); human amniotic mesenchymal stromal cells (hAMSC); human chorionic mesenchymal stromal cells (hCMSC); and human chorionic trophoblastic cells (hCTC).

The term “amnion” refers to a membranous sac that surrounds and protects the embryo. Its primary function is the protection of the embryo for its future development into a fetus and eventually an animal. The amnion is the inner of the two fetal membranes surrounding the fetus (the chorion is the outer one). The terms “amnion”, “amniotic membrane”, and “amniotic tissue” are all used interchangeably in the present application. The amnion may be obtained from any reptilian, avian or mammalian species including rodents, humans, non-human primates, equines, canines, felines, bovines, porcines and the like. Preferably the amnion of the present application is obtained from human.

The term “amnion epithelial cell” is used synonymously herein with the term “amnion epithelial stem cell”, “hAEC”, and “AEC”. Amnion epithelial cells as used herein refer to cells that are obtained from the amnion, specifically the inner layer of epithelial cells.

The term “amnion mesenchymal cells” is used synonymously with “amnion mesenchymal stem cells”, “amnion mesenchymal stromal cells”, “hAMC”, and “AMC”. Amnion mesenchymal cells as used herein refer to cells that are obtained from the amnion, specifically the outermost layer of the amnion juxtaposed to the chorion.

The term “differentiation” refers to the structure or function of cells becoming specialized during division, proliferation and growth thereof, that is, the feature or function of a cell or tissue of an organism changes in order to perform work given to the cell or tissue.

The term “neural cells” are cells having at least an indication of neuronal or glial phenotype, such as staining for one or more neuronal or glial markers or which will differentiate into cells exhibiting neuronal or glial markers. Examples of neuronal markers that may be used to identify neuronal cells according to the present invention include, for example, neuron-specific nuclear protein, tyrosine hydroxylase, microtubule associated protein, and calbindin, among others. The term neural cells also includes cells which are neural precursor cells, i.e., stem and/or progenitor cells which will differentiate into or become neural cells or cells which will ultimately exhibit neuronal or glial markers, such term including pluripotent stem and/or progenitor cells, including but not limited to placental derived stem cells such as amnion derived epithelial cells and amnion derived mesenchymal stem cells, which ultimately differentiate into neuronal and/or glial cells. All of the above cells and their progeny are construed as neural cells for the purpose of the present invention. The terms “neural cells” and “neuronal cells” are generally used interchangeably in many aspects of the present invention. Preferred neural cells for use in certain aspects according to the present invention include those cells which exhibit one or more of the neural/neuronal phenotypic markers such as Musashi-1, Nestin, NeuN, class III -tubulin, GFAP, NF-L, NF-M, microtubule associated protein (MAP2), S100, CNPase, glypican (especially glypican 4), neuronal pentraxin II, neuronal PAS 1; neuronal growth associated protein 43, neurite outgrowth extension protein, vimentin, Hu, internexin, 04, myelin basic protein, TuJ1, and pleiotrophin, among others.

“Administration” or “administering” is used to describe the process in which a compound or combination of compounds of the present invention are delivered to a patient. The composition may be administered in various ways including parenteral (referring to intravenous and intraarterial and other appropriate parenteral routes), intratheceal, intraventricular, intraparenchymal (including into the spinal cord, brainstem or motor cortex), intracranial, intrastriatal, intracisternal, intranigral, among others which term allows cells of the subject invention to migrate to the ultimate site where needed. Each of these conditions may be readily treated using other administration routes of compound or any combination of compounds thereof to treat a disorder or condition. The compositions according to the present invention may be used without treatment with a mobilization agent or differentiation agent (“untreated” i.e., without further treatment in order to promote differentiation of cells within the stem cell sample) or after treatment (“treated”) with a differentiation agent or other agent which causes certain stem and/or progenitor cells sample to differentiate into cells exhibiting a differentiated phenotype, such as a neuronal and/or glial phenotype.

Administration will often depend upon the disease or condition treated and may preferably be via a parenteral route, for example, intravenously, by administration into the cerebral spinal fluid or by direct administration into the affected tissue in the brain. For example, in the case of Alzheimer's disease, Huntington's disease, and Parkinson's disease, the preferred route of administration will be a transplant directly into the striatum (caudate cutamen) or directly into the substantia nigra (Parkinson's disease). In the case of amyotrophic lateral sclerosis (Lou Gehrig's disease) and multiple sclerosis, the preferred administration is through the cerebrospinal fluid. In the case of lysosomal storage disease, the preferred route of administration is via an intravenous route or through the cerebrospinal fluid. In the case of stroke, the preferred route of administration will depend upon where the stroke is, but may be directly into the affected tissue (which may be readily determined using MRI or other imaging techniques), or may be administered systemically. In a preferred embodiment of the present invention, the route of administration for treating an individual post-stroke is systemic, via intravenous or intra-arterial administration.

The terms “grafting” and “transplanting” and “graft” and “transplantation” are used throughout the specification synonymously to describe the process by which cells of the subject invention are delivered to the site where the cells are intended to exhibit a favorable effect, such as repairing damage to a patient's central nervous system (which can reduce a cognitive or behavioral deficit caused by the damage), treating a neurodegenerative disease such as Alzheimer' s disease or Parkinson's disease, or treating the effects of nerve damage caused by stroke, cardiovascular disease, a heart attack or physical injury or trauma or genetic damage or environmental insult to the brain and/or spinal cord, caused by, for example, an accident or other activity. Cells of the subject invention can also be delivered in a remote area of the body by any mode of administration as described above, relying on cellular migration to the appropriate area to effect transplantation.

“Disease model” is defined as any scientifically accepted means of inducing a disease condition in vitro, including but not limited to oxygen glucose deprivation (OGD) as a stroke model and oxidative stress as a stroke model or Parkinson model by administration of H202.

“Oxidative Stress” refers to an imbalance between the production of reactive oxygen and a biological system's ability to readily detoxify the reactive intermediates or easily repair the resulting damage. Oxidative stress produces reactive oxygen species including, but not limited to, free radicals and peroxides. Oxidative stress has been implicated in many diseases including but not limited to atherosclerosis, stroke, ischemia, Alzheimer' s disease, Parkinson's disease, myocardial infarction, Huntington's disease, amyotrophic lateral sclerosis (ALS) and chronic fatigue syndrome, among others. Oxidative stress is induced in a disease model through the administration of H202 to the cells which induces a stroke-like state.

The term “acute neurodegenerative disease” means and disease or disorder associated with an abrupt insult, resulting in associated neuronal death or compromise. Exemplary acute neurodegenerative diseases include cerebrovascular insufficiency, focal or diffuse brain trauma, spinal cord injury, cerebral ischemia or infarction, including emolic occlusion and thrombotic occlusion, perinatal hypoxic-ischemia, neonatal hypoxia-ischaemic encephalopathy, perinatal asphyxia, cardiac arrest, intracranial hemorrhage, stroke, and traumatic brain injury.

The term “neurodegenerative disease” is used herein to describe a progressive or chronic disease which is caused by damage to the central nervous system and which damage can be reduced and/or alleviated through transplantation of amnion derived cells according to the present invention directly into, but preferably via systemic route that will allow the cells or their soluble factors to reach the damaged areas of the brain and/or spinal cord of the patient. Exemplary neurodegenerative diseases which may be treated using the neural cells and methods according to the present invention include for example, Parkinson's disease, Huntington's disease, amyotrophic lateral sclerosis, Alzheimer's disease, Rett Syndrome, lysosomal storage diseases (“white matter disease” or glial demyelination disease, as described, for example by Folkerth, J. Neuropath. Exp. Neuro., September 1999, 58:9), including Sanfillippo, Gaucher disease, Tay Sachs disease (beta hexosaminidase deficiency), other genetic diseases, multiple sclerosis, brain injury or trauma caused by ischemia, accidents, environmental insult, etc., spinal cord damage, ataxia and alcoholism. In addition, the present invention may be used to reduce and/or eliminate the effects on the central nervous system of a stroke or a heart attack in a patient, which is otherwise caused by lack of blood flow or ischemia to a site in the brain of said patient or which has occurred from physical injury to the brain and/or spinal cord. Neurodegenerative diseases also include neurodevelopmental disorders including for example, cerebral palsy, autism and related neurological diseases such as schizophrenia, among numerous others.

“Melatonin Receptor 1” is used synonomously with MT1 and MelR1 throughout this application and is described as a G protein coupled receptor that binds melatonin. MelR1 is found mainly in the pars tuberalis of the pituitary gland and the suprachiasmic nuclei of the hypothalamus. The inventors discovered that amnion derived cells are MelR1 positive cells, indicating that these cells possess this specific melatonin receptor.

“Melatonin Receptor 2” is used synonomously with MT2 and MelR2 throughout this application and is described as a G protein coupled receptor that binds melatonin. MelR2 is found mainly in the retina in humans.

“Melatonin” refers to the chemical compound N-acetyl-5-methoxytryptamine. Melatonin is produced by many parts of the body including, but not limited to, the pineal gland, the retina, the gastrointestinal tract, epithelial cells, bone marrow cells and lymphocytes. Melatonin can also be manufactured in the lab for administration to mammals and is readily available for commercial use. Melatonin acts as an antioxidant that can easily cross cell membranes and the blood brain barrier and is a direct scavenger of OH, O₂⁻ and NO.

Stem cells have been considered as potential treatments for various debilitating diseases including cardiovascular disease, stroke and Parkinson's disease. Stem cells have the potential to develop into many different cell types in the body and can theoretically divide without limit to replenish other cells. When a stem cell divides, each new cell has the potential to remain a stem cell or to become another type of cell with a more specialized function such as a muscle cell or nerve cell.

Stem cells are often characterized as totipotent, pluripotent or multipotent. Totipotent stem cells (e.g. zygote) give rise to both the fetus and the extraembryonic tissues. Pluripotent stem cells can give rise to any type of cell except for the extraembryonic tissues (e.g. placenta). Multipotent stem cells can give rise to two or more different cell types but only within a given organ or tissue type. In contrast to stem cells, progenitor cells are unable to self-renew and can only give rise to a few cell types.

### Placental Derived Cells

The development of cell therapy approaches using placenta-derived cells can benefit from the fact that placental tissues harbor different cell types that may complement each other in a clinical setting (i.e., amniotic epithelial cells of early embryological origin with multilineage differentiation potential, as well as cells with immunomodulatory properties). Aside from being easily procured in a painless and noninvasive manner, placental cells also offer additional advantages over stem cells from other sources such as bone marrow, which carry a risk of viral infection, and also show decreasing differentiation capacity with increasing donor age. Placenta-derived cells may also be preferable from an immunological point of view, given the unique role of this tissue in maintaining fetomaternal tolerance throughout pregnancy. Placental cells show a greater capacity to down-regulate T-cell proliferation in vitro compared to bone marrow-derived cells. Placenta-derived cells have been investigated for their potential to confer beneficial effects in a range of neurological disorders. (Parolini, 2010)

Placenta-derived stem cells are further divided into human amniotic epithelial cells (hAEC); human amniotic mesenchymal stromal cells (hAMSC); human chorionic mesenchymal stromal cells (hCMSC); and human chorionic trophoblastic cells (hCTC). (Parolini, 2010)

### Amnion Derived Epithelial Cells

Amniotic membrane, or amnion, has recently emerged as another novel and alternative fetal source of stem-cell populations. Specifically, amniotic membrane, lacking any vasculature, is derived from the epiblast by day 8, comprising three layers of which are an inner epithelial layer consisting of epithelial cells (AECs); an intermediate basement membrane lacking any cellular component; and an outer layer juxtaposed to the chorion consisting of mesenchymal cells called amniotic mesenchymal or amniotic mesenchymal stromal cells (AMCs). Since these amnion cells, often called amnion-derived stem cells, originate from epiblast cells, it is conceivable that they might retain, and eventually portray, several stem-cell features through gestation and are associated with a low percentage of HLA antigen-expressing cells. Primary AECs seem to contain class 1A and class IIHLAs, consistent with a low risk of tissue rejection. (Pappa, K. et al., Novel Sources of Fetal Stem Cells: Where Do They Fit on the Developmental Continuum?, Regen. Med., 2009; 4 (3): 423-433)

Characterization of hAEC has shown that these cells express molecular markers of pluripotency and can differentiate in vitro into cell types of all three germ layers. (Parolini 2010) These amnion stem cells do not generate teratomas in vivo in contrast to ESCs. (Pappa 2009) These properties, the ease of isolation of the cells, and the availability of placenta as a discard tissue, make the amnion a potentially useful and noncontroversial source of cells for transplantation and regenerative medicine. (Parolini 2007)

Amniotic membrane contains epithelial cells with different surface markers, suggesting some heterogeneity of phenotype. Immediately after isolation, hAEC appear to express very low levels of HLA- A,B,C, however, by passage 2, significant levels are observed. Additional cell surface antigens on hAEC include ATP-binding cassette transporter G2 (ABCG2/BCRP), CD9, CD24, E-cadherin, integrins alpha 6 and beta 1, c-met (HGF receptor), stage specific embryonic antigens (SSEA) 3 and 4 and tumor rejection antigens (TRA) 1-60 and 1-81. Surface markers thought to be absent on hAEC include SSEA-1, CD34, and CD133, while other markers such as CD117 (c-kit) and CCR4 (CC chemokine receptor) are either negative or may be expressed on some cells at very low levels. Although initial cell isolates express very low levels of CD90 (Thy-1), the expression of this antigen increases rapidly in culture. (Parolini 2007)

In addition to surface markers, hAEC express molecular markers of pluripotent stem cells, including octamer-binding-protein-4 (OCT-4), SRY-related HMG-box gene 2 (SOX-2), and Nanog. Studies have indicated that differentiation of hAEC can be directed and that cultured hAEC synthesize and release acetylcholine, catecholamines and dopamine. Cell types from all three germ layers have been produced in vitro. There is currently strong in vitro and in vivo evidence of neural, pancreatic and hepatic differentiation of hAEC. (Parolini 2007)

Human AEC have shown particular potential for treating central nervous system disorders. Since the discovery that hAEC have stem cell properties, express neural and glial markers and neural-specific proteins, and also have the capacity to produce and secrete neurotransmitters, cell therapy with these cells has been considered. Successful transplants of hAEC into caudate nucleus, hippocampus and spinal cord have been reported. Transplantation of hAEC in a rat model of Parkinson's reversed the condition and prevented neuronal death. When hAEC were transplanted into ischemic hippocampus, they differentiated into “neuronlike” cells. Following transplantion into the transected spinal cord of monkeys, hAEC aided a robust regeneration of host axons and prevented death of axotomised neurons of the spinal cord. Transplantation of hAEC into the lesioned areas of a contusion model of Spinal Cord Injury (SCI) in rats was performed without immunosuppression. Cells survived up to 120 days with no evidence of inflammation or rejection. Animals showed gradual functional improvement using the Basso, Beattie and Brensnahan (BBB) locomotor rating scale, and ultimately reached a score of 19, just two points below normal animals. Improvement was also observed in control animals, however, improvement was faster during acute and sub-acute phases of recovery in transplant recipients. Early improvement in the BBB scale is thought to indicate that hAEC provide neuroprotection. Human AEC secrete neurotrophic factors, while medium conditioned by hAEC has been shown to be neurotrophic for E18 rat cortical cells. Novel EGF-like neurotrophic factors were thought to mediate this effect. hAEC conditioned medium also supported survival of E10 chicken neural retinal cells, which were otherwise dependent on fibroblast growth factor-2 (FGF-2). Although FGF-2 and EGF were not detected in media by immunoblotting, FGF-2 and EGF gene and protein expression was reported in cryopreserved hAEC. hAECs were found to exhibit neuroprotection in acute phases of injury and facilitate regeneration of long tracts in longterm phases of recovery, as measured by behavioral assessment. The beneficial effects may be mediated through the secretion of novel neurotrophic factors. (Parolini, O. et al., Isolation and Characterization of Cells from Human Term Placenta: Outcome of the First International Workshop on Placenta Derived Stem Cells, Stem Cells Express, Nov. 8, 2007 p. 1-11)

In preclinical studies using animal models of Parkinson's disease and ischemia, hAEC have been found to offer neuroprotection and functional recovery. The observed therapeutic effects are likely mediated by secretion of diffusible factors, including neurotransmitters and many neurotrophic and growth factors. (Parolini 2010) With regard to stroke, because inflammation is a major contributor to the secondary cell death cascade following the initial stroke episode, transplanted cell-mediated abrogation of such inflammatory deleterious side effects should directly alter stroke progression. A major caveat for this anti-inflammatory mechanism to effectively mitigate cell therapy and stroke outcome is demonstrating robust and stable secretion of anti-inflammatory factors by transplanted cells at the appropriate timing post-injury. Although inflammation is shown to exacerbate stroke, early pathological inflammatory cues, such as stromal derived factor-1, serve as a migratory guide for transplanted cells to reach the ischemic tissue. (Parolini 2010)

Cell therapy has been proposed as a novel treatment for acute, subacute, and chronic stroke. Transplantation of human placenta-derived cells has been shown to exert beneficial effects in a rodent stroke model. Specifically, transplantation of hAEC or hAMSC at Day 2 post-stroke attenuated both motor and neurological deficits associated with occlusion of the middle cerebral artery at days 7 and 14 compared to the vehicle-infused stroke group. Following the last behavioral test at Day 14 post-stroke, histology via Nissl staining revealed transplantation of hAEC or hAMSC at Day 2 post-stroke increased the number of healthy looking cells (>75% of the intact brain) in the ischemic penumbra compared to the vehicle-infused stroke group. These positive behavioral and histological effects were achieved when 400,000 human placenta cells were transplanted directly into the presumed ischemic penumbra in the absence of immunosuppression. (Parolini, O. et al. (2010) Toward Cell Therapy Using Placenta-Derived Cells: Disease Mechanisms, Cell Biology, Preclinical Studies, and Regulatory Aspects at the Round Table, STEM CELLS AND DEVELOPMENT, Vol. 19, No. 2:143-154)

It has recently been found that the treatment of cultured AECs with various differentiation factors effectively promotes neuronal marker expression. Treatment of AECs with differentiation agents such as Noggin and retinoic acid increased the number of cells expressing neuronal markers. RA application induced concentration dependent differentiation of neural cells. Concomitant treatment of cells with RA and bFGF produced the highest level of neural marker expression. (Niknejad, H. et al., (2010) Differentiation Factors that Influence Neuronal Markers Expression In Vitro from Human Amniotic Epithelial Cells, European Cells and Materials, Vol. 19:22-29)

### Amnion Derived Mesenchymal Cells

hAMSC can be isolated from first-, second- and third-trimester mesoderm of amnion and chorion, respectively. For hAMSC, isolations are usually performed with term amnion dissected from the deflected part of the fetal membranes to minimize the presence of maternal cells. Homogenous hAMSC populations can be obtained by a two-step procedure: minced amnion tissue is treated with trypsin to remove hAEC, and remaining mesenchymal cells are then released by digestion with collagenase, or collagenase and DNase. The yield from term amnion is about 1 million hAMSC and 10-fold more hAEC per gram of tissue.

hAMSC adhere and proliferate on tissue culture plastic, and can be kept until passage 5-10. Reports suggest that hAMSC proliferation slows beyond passage 2, although first-trimester hAMSC proliferate better than third-trimester cells. Theoretically, term amnion may yield up to 5×10⁸ hAMSCs, however in practice, yields are typically 4 million hAMSC/100 cm2 starting material with a 4-fold expansion after one-month (2 passages). (Parolini 2007)

The hAMSC show multilineage differentiation potential. Mesenchymal cells from the amniotic and chorionic membranes also have the ability to differentiate in vitro into a range of neuronal and oligodendrocyte precursors. (Parolni 2010) The plasticity of amnion-derived stem cells has also been recently tested in cultures at the clonal level, where long term self-renewal and multidifferentiation capacity have been documented. The proliferation rate of AM-MSCs was found to lead to an approximately 300-fold expansion in 21 days, yielding 2.9×10⁶ cells. The outer layer of amniotic membrane has recently been shown to represent a rich source for MSCs with the ability to differentiate into endothelial cells in vitro or to cardiocytes and hepatocytes in vitro and in vivo. (Pappa 2009)

Another important feature of human AM-MSCs and of human epithelial cells is their ability to exhibit a contact-and dose-dependent immunomodulatory effect on peripheral blood mononuclear cells. This property reflects a general capacity of MSCs or stromal cells derived from different sources and it seems to be mediated via a mechanism involving the release of nitric oxide by MSCs in response to proinflammatory cytokines following T cell activation. (Pappa 2009)

### Melatonin

The central nervous system (CNS) is especially vulnerable to free radical damage because of brain's high oxygen consumption, its abundant lipid content, and the relative paucity of antioxidant enzymes as compared with other tissues. Moreover, the brain has a high ratio of membrane surface area to cytoplasmic ratio, extended axonal morphology prone to injury, and neuronal cells that are non-replicating. ROS can increase the permeability of the blood brain barrier, alter tubulin formation, and inhibit the mitochondrial respiration. If left unchecked, it can lead to a geometrically progressing lipid peroxidation. Evidence also indicates that ROS may stimulate extracellular release of excitatory amino acids. Glutamate is the major excitatory amino acid in the brain. It acts through various types of ionotropic receptors, the most significant being, NMDA receptors. There seems to be a bi-directional relationship between the ROS production and release of excitatory amino acids. Free radicals generated in the brain are also reported to influence gene expression, subsequently effecting apoptosis and neuronal death. (Gupta, 2003)

Oxidative stress has been implicated in various neurological disorders, such as epilepsy, Alzheimer's disease, Parkinson's disease, stroke, cerebral ischemia, multiple sclerosis, Huntington's chorea, tardive dyskinesia, and amyotrophic lateral sclerosis etc. The brain is deficient in oxidative defense mechanisms and hence is at greater risk of damage mediated by reactive oxygen species (ROS) resulting in molecular and cellular dysfunction. Emerging evidence suggesting the activation of glutamate gated cation channels, may be another source of oxidative stress, leading to neuronal degeneration. (Gupta, 2003)

The term ‘oxidative stress’ refers to the imbalance between oxygen species (ROS) and the antioxidant opposing forces. ROS may be oxygen centered radicals possessing unpaired electrons such as superoxide dismutase anion and hydroxyl radical, or covalent molecules such as hydrogen peroxide. The fact that oxygen is ubiquitous in aerobic organisms has led to the concept of the oxygen paradox; namely the fact that this life supporting molecule is also a precursor to the formation of harmful reactive oxygen species (ROS). ROS can damage virtually any biological molecule in its vicinity including DNA, essential proteins, and membrane lipids. (Gupta, 2003)

Melatonin, the pineal hormone, acts as a direct free radical scavenger and indirect antioxidant. The importance of melatonin as an antioxidant depends on several characteristics: its lipophilic and hydrophilic nature, its ability to cross all barriers with ease, and its availability to all tissues and cells. It distributes in all cellular compartments, being especially high in the nucleus and mitochondria. Tissues except pineal gland producing melatonin for local use include the retina, cells of the immune system, bone marrow, human ovary, lens and testes. Levels of melatonin are two to three orders of magnitude higher than maximal blood melatonin concentrations in cerebrospinal fluid (CSF). Melatonin has been shown to either stimulate gene expression for the antioxidant enzymes (superoxide dismutase, catalase, glutathione peroxidase, glutathione reductase) or to increase their activity. Additionally, it neutralizes hydoxyl radical, superoxide radical, peroxyl radical, peroxynitrite anion, singlet oxygen, hydrogen peroxide, nitric oxide, and hypochlorous acid. Unlike other antioxidants, melatonin can easily cross all morphophysiological barriers, e.g., the blood brain barrier, and enters cells and subcellular compartments. (Gupta, 2003)

In the brain, an array of cellular defense systems exists to counterbalance the ROS. These include enzymatic and nonenzymatic antioxidants that lower the concentration of free radical species and repair oxidative cellular damage. Glutathione functions as a major antioxidant in tissue defense against free radicals in the brain. The brain is known to synthesize molecules like glutathione and NADPH. But, the concentration of glutathione is relatively in lesser quantities in the brain as compared to the rest organs of the body. The natural antioxidant system present in brain can be in the form of enzymes like catalase, peroxidase, superoxide dismutase or low molecular weight antioxidants. Low molecular weight antioxidants can be ascorbic and lipoic acids, carotenoids or indirectly acting, like chelating agents. (Gupta, 2003)

Melatonin is ubiquitously present endogenously in brain. Its concentrations have been found to be raised after seizures and altered in neurological conditions. Melatonin has a wide safety margin and is known to cross the blood brain barrier. The experimental studies have shown the effectiveness of melatonin in Parkinsonism, epilepsy, stroke, Alzheimer's disease, movement disorders etc. Melatonin has a half life of nearly 30-53 minutes, and no significant side effects except sedation in high doses in experimental studies are known. These characteristics make it an attractive candidate to be therapeutically exploited in chronic conditions. The function of melatonin as antioxidant and free radical scavenger is facilitated by the ease with which it crosses morphophysiological barriers like blood brain barrier, intracellular and subcellular barriers. (Gupta, 2003)

Melatonin is well known as a regulator of biological rhythms by controlling the phase and amplitude of circadian rhythm by acting both on suprachiasmatic nucleus (SCN), the biological clock that resides in the hypothalamus as well as on various other cells and tissues of the body. Melatonin likely works via electron donation to directly detoxify free radicals. In in-vitro and in-vivo experiments, melatonin has been found to protect cells, tissues and organs against oxidative damage induced by a variety of free radical generating agents and processes, including cyanide poisoning, glutathione depletion, ischemia reperfusion, kainic acid induced excitotoxicity, and 1-methyl-4-phenyl- 1,2,3,6-tetrahydropyridine (MPTP). Melatonin as an antioxidant is not only effective in protecting nuclear DNA, membrane lipids and possibly cytosolic proteins from oxidative damage but is also reported to alter the activities of enzymes that improve the total antioxidative defense capacity of the organism. (Gupta, 2003)

Melatonin scavenges the —OH resulting in the formation of cyclic 3 hydroxymelatonin, a harmless product that is excreted in the urine, which also acts as a free radical scavenger. Each molecule of melatonin scavenges two —OH, unlike other antioxidants, which lack the ability to quench the hydroxyl radicals. Unlike other well known antioxidants that are exclusively lipid (e.g. vitamin E) or water soluble (e.g. vitamin C) and therefore, exhibit a limited intracellular distribution, Melatonin is amphiphilic allowing it to reduce —OH mediated damage in both the lipid and aqueous subcellular compartments. It has recently been discovered that melatonin also directly neutralizes the precursor of —OH, namely hydrogen peroxide (H202). When melatonin scavenges H202 the product has been identified as N1 acetyl N2 formyl 5 methoxykynuramine (AFMK), which in addition is shown to be capable of donating two electrons and, therefore being a direct free radical scavenger in its own right. This phenomenon is referred to as the antioxidant cascade where melatonin as well as at least one resulting metabolite are both highly effective scavengers. (Gupta, 2003)

Steady state levels of ONOO— are reduced when melatonin scavenges nitric oxide (NO). NO normally couples with O2 to form ONOO—. Melatonin also reduces the generation of NO— by inhibiting the activity of its rate limiting enzyme, nitric oxide synthase (NOS). In in-vivo studies where melatonin's efficacy was compared with classical antioxidants in terms of pharmacologically protecting against free radical damage, melatonin was found to be effective at a lower dose than other antioxidants. (Gupta, 2003)

### Melatonin In Alzheimer's Disease

In recent years considerable data has been generated indicating that the brain in Alzheimer's Disease (AD) is under oxidative stress, and this may have a role in the pathogenesis of neuron degeneration and cell death in this disorder. Increased oxidative stress in AD shows increased brain iron, aluminum, and magnesium. In the brain, these are capable of stimulating free radical generation, lipid peroxidation and PUFA, protein and DNA oxidation, diminishing energy metabolism, advanced glycation end products (AGE), MDA, SOD-1 in senile plaques. Studies have shown that amyloid beta peptide is capable of generating free radicals. Melatonin has been reported to inhibit the formation of β amyloid protein from its precursor and reduce aluminum ion-induced peroxidation. It has recently been reported that melatonin and pinoline reduced, in a concentration dependent manner, lipid peroxidation due to aluminum, FeCl3 and ascorbic acid in the synaptosomal membranes.

### Melatonin In Cerebral Ischemia

Acute ischemic stroke is the third largest cause of mortality and is the single largest cause of adult disability. The present therapeutic approaches in stroke are primarily vascular (reperfusion) or neuronal (neuroprotection). The perplexing problem with the reperfusion is the massive generation of free radicals, which starts the cascade of events leading to neuronal death. Realizing this, the role of antioxidants in stroke is being widely researched. Free radical generation during cerebral ischemia may underlie delayed neuronal death. It has been proposed that during ischemia, ROS and excitatory amino acids may cooperate in neuronal damage. Transient ischemia elevates cerebral levels of both excitatory amino acids and rates of hydroxyl radical formation. Melatonin treatment has been shown to be highly effective in different in vivo and in vitro models of excitotoxicity or ischemia/reperfusion in multiple animal species. (Gupta, 2003)

Since melatonin is endogenously produced, the organisms have evolved mechanisms to remove excessive amounts from the body. Virtually all exogenously administered antioxidants have a dose at which they become toxic. Melatonin, even when given in massive amounts (300 mg daily) for prolonged periods (up to 5 years) to humans has not produced untoward side effects. It has been used in doses of 3 mg to 300 mg in clinical trials. The bulk of the studies that have tested the antioxidant capacity of melatonin have used pharmacological doses. (Gupta, 2003)

A number of studies have shown that surgical removal of the pineal gland leads to exaggerated free radical damage. For example, when compared to intact rats, pinealectomized animals exhibited much greater free radical based neural damage induced by ischemia-reperfusion. Furthermore in rats as well as in humans, blood levels of melatonin positively correlate with the ability of this fluid to detoxify free radicals. (Gupta, Y. K., et al., Neuroprotective Role of Melatonin in Oxidative Stress Vulnerable Brain, Indian J Physiol Pharmacol (2003); 47 (4): 373-386)

Administration of melatonin exhibits a bi-phasic response which is typical to antioxidants, which at higher doses may, via interaction with other oxidants or antioxidants, turn into pro-oxidants. In the early post-injury phase, melatonin may directly neutralize excess ROS, thereby leading to attenuated consumption of other endogenous antioxidants. Under prolonged oxidative stress, melatonin may potentiate tissue antioxidants via distinct, time-dependent mechanisms, such as induction of antioxidant enzymes and/or inhibition of pro-oxidant enzymes. Overproduction of ROS occurs within minutes after brain injury and mediates both necrotic and apoptotic cell death. In addition, H202 leads to the activation of protein tyrosine-kinases followed by the stimulation of downstream signaling pathways including mitogen-activated protein kinases and phospholipase C. Such reactions, in concert, result in the activation of redoxsensitive transcription-factors, including NF-κB and AP-1. Oxidative stress is the result of imbalance between ROS production and elimination and could be viewed as a threshold phenomenon that occurs after endogenous antioxidant mechanisms are overwhelmed. It has been suggested that neuroprotection via melatonin is mediated via potentiation of other brain antioxidants (e.g., ascorbic acid, and other, yet unidentified compounds), thus altering the redox state of the cell and consequently attenuating NF-κB and AP-1 activation. (Beni, S. M. et al., Melatonin-Induced Neuroprotection After Closed Head Injury Is Associated with Increased Brain Antioxidants and Attenuated Late-Phase Activation of NF-κB and AP-1, The FASEB Journal, 2003)

Neuroprotective effects of melatonin have been demonstrated mainly in models of neuronal cell death in which oxygen free radicals or excitotoxins are involved. In the N-methyl- 4-phenylpyridinium and 6-hydroxydopamine (6-OHDA) models of Parkinson's disease, melatonin completely reversed the rises in lipid peroxidation products, the decrease in tyrosine hydroxylase in striatum and substantia nigra, and rescued dopamine neurons in culture. Melatonin also prevented kainate-induced neuronal cell death and reduced lipid peroxidation products in rats and mice in vivo. Furthermore, melatonin protects against glutamate-induced cell death in the clonal hippocampal cell line HT22, prevents delayed neuronal death induced by enhanced excitatory transmission in hippocampal pyramidal neurons in culture, and rescues neuroblastoma cells exposed to toxic fragments of Alzheimer's β-amyloid. An anticonvulsant activity of melatonin has been demonstrated against excitotoxin-induced seizures by quinolinate, kainate, and glutamate in mice and by iron or amygdala kindling in rats. The occurrence of increased brain damage after stroke or excitotoxic seizures in melatonin-deficient rats is in line with these findings. Besides the antioxidant potential, several other mechanisms are considered to be involved in the neuroprotection mediated by melatonin, including interactions with calmodulin and microtubular components, blockade of increases in intracellular Ca21 levels, maintenance of cellular glutathione homeostasis, inhibition of activation of NF-κB by cytokines such as tumor necrosis factor α, inhibition of the expression of inducible nitric oxide synthase at the transcriptional level, and changes in gene expression of antioxidant enzymes. Melatonin attenuates neuronal apoptosis in the case of 6-OHDA- , b-amyloid-, and kainate-induced cell damage in vitro and in vivo. In vivo evidence is available that melatonin protects against DNA damage, which was observed in the hippocampus 48 and 72 h after intraperitoneal administration of kainate to rats. Up-regulation of the glutathione antioxidative defense system by melatonin has been suggested as a mechanism for reducing neuronal death caused by excitotoxicity and for preventing the kainate-induced damage from spreading to adjacent brain regions. Melatonin is believed to work via electron donation in detoxifying the —OH radical. Melatonin is considered an endogenous neuroprotective factor useful for the pharmacological treatment of neurodegeneration produced by glutamate excitotoxicity and/or oxidative stress, such as brain ischemia or epilepsia. (Harms, C. et al., Melatonin is protective in Necrotic but not in Caspase-Dependent, Free Radical-Independent Apoptotic Neuronal Cell Death in Primary Neuronal Cultures, The FASEB Journal, 2000; 14: 1814-1824)

The mammalian MT1 receptor contains two glycosylation sites in its N-terminal. There is increasing evidence that melatonin is involved in the early development of vertebrates. Melatonin is produced in chick embryos as early as the 7^{th} day of embryonic development and a physiological concentration of this hormone has been shown to significantly enhance mouse embryogenesis in vitro. Various studies have found that melatonin receptors are present in the human fetal brain and peripheral tissues. Recent audioradiographic and in situ hybridization studies indicate that the melatonin MT1 receptor is expressed in diverse areas of the human fetal brain. MT1 receptors have also been seen in neural and glial progenitor cells, which is consistent with a neurodevelopmental role for melatonin and suggests that in addition to the presence of the MT1 in mammalian neurons, it may also be expressed in astrocytes. (Niles, L. et al., Neural Stem Cells Express Melatonin Receptors and Neurotrophic Factors: Colocalization of the MT1 Receptor with Neuronal and Glial Markers, BMC Neuroscience (2004) 5:41)

As referenced above, the human placenta is a good source of stem cells. The inventors have found that transplantation of these human placenta-derived cells in an in vivo stroke model promotes functional recovery through the release of soluble factors. The inventors have discovered that placenta-derived stem cells express one (MelR1) of two discrete types of melatonin receptors. Stimulation of this receptor on amnion epithelial stem cells (AECs) with the administration of melatonin resulted in a synergistic/additive neuroprotective effect.

The inventors herein discovered that the therapeutic benefits of stem cells are produced following disease induction and activation of a receptor. The stem cells used herein were derived from human amnion. In particular, it was shown that cultured stem cells exposed to an in vitro model of stroke, called oxygen glucose deprivation (OGD), secrete high levels of trophic factors compared to stem cells grown in ambient condition (i.e., appropriate oxygen and glucose supplementation).

ELISA revealed high levels of VEGF and GDNF in the conditioned media from

OGD-exposed stem cells. Negligible levels of trophic factors were detected in non-OGD-exposed stem cells. Equally novel, it was shown that the majority of these cultured stem cells express the melatonin receptor 1 (MelR1), but not melatonin receptor 2 (MelR2). Furthermore, treatment of these cultured stem cells with the ligand melatonin, at specific doses, display decreased proliferation but increased differentiation into a neural lineage.

To reveal the neuroprotective effects of these stem cells, cultured primary rat cells (gestation age 18) were initially exposed to OGD and immediately thereafter stem cells or conditioned media (harvested from OGD-exposed stem cells over 7 days) were added. Parallel sister cultures included non-OGD exposed rat cells (positive control). Stem cell doses were varied at 0 (negative control), 2.5%, 5%, 10% or 25% of total cell population per well. After 3 hours of stem cell or conditioned media treatment, cell viability (Trypan blue and MTT assay) and ELISA were performed.

It was found that treatment with stem cells significantly reduced cell death in OGD-exposed rat cells in a dose-dependent manner. Interestingly, conditioned media from OGD-exposed stem cells also exerted significant amelioration of OGD-induced cell death comparable to that produced by the stem cell treatment.

In order to demonstrate the role of melatonin receptor 1, stem cells were treated with melatonin and showed a dose-dependent suppression of proliferation coupled with dose-dependent enhancement of neural differentiation. Altogether, these data reveal that therapeutically active substances released by stem cells and melatonin receptor activation in the stem cells principally contribute to neuroprotective effects against cell death.

### EXAMPLE 1

### Soluble Factors Release By Human Placenta-Derived Cells Mediate Neuroprotection In Stroke Model

Recent studies have demonstrated that human placenta is a good source of stem cells. The inventors have provided laboratory evidence that transplantation of these human placenta-derived cells in an in vivo stroke model promotes functional recovery. The inventors have discovered that soluble factors released by these transplanted cells mediated the therapeutic benefits.

The human amnion was provided by Dr. Parolini under approved institutional guidelines. Subsequent cell culture and transplant experiments on the human amnion were conducted at the collaborating US research institution under approved protocols. The embryonic stem cell phenotypic marker Oct-4 was used to reveal the stemness of Amniotic Epithelial Cells (AECs) and Amniotic Mesenchymal Cells (AMCs). Cultured primary rat cells (gestation age 18) were initially exposed to the oxygen glucose deprivation (OGD) injury model (92% N2 and 8% O2 gas for 90 minutes), and immediately thereafter AECs, AMCs or conditioned media (harvested from AECs or AMCs cultured over 7 days) were added to the OGD-exposed cells. Parallel sister cultures included non-OGD exposed rat cells (positive control). Placenta cell doses were varied at 0 (negative control), 2.5%, 5%, 10% or 25% of total cell population per well. After 3 hours of placenta cell or conditioned media treatment, cell viability (Trypan blue and MTT assay) and ELISA were performed.

Treatment with AECs and AMCs significantly reduced cell death in OGD-exposed rat cells in a dose-dependent manner, with no discernable difference in neuroprotective effects between the two placenta cell types. Interestingly, conditioned media from AECs and AMCs also exerted significant amelioration of OGD-induced cell death comparable to that exerted by the placenta cell treatment. ELISA revealed high levels of VEGF and GDNF in the conditioned media from both AECs and AMCs.

These results reveal that therapeutically active substances released by human placenta-derived cells principally contribute to neuroprotective effects against ischemic cell death.

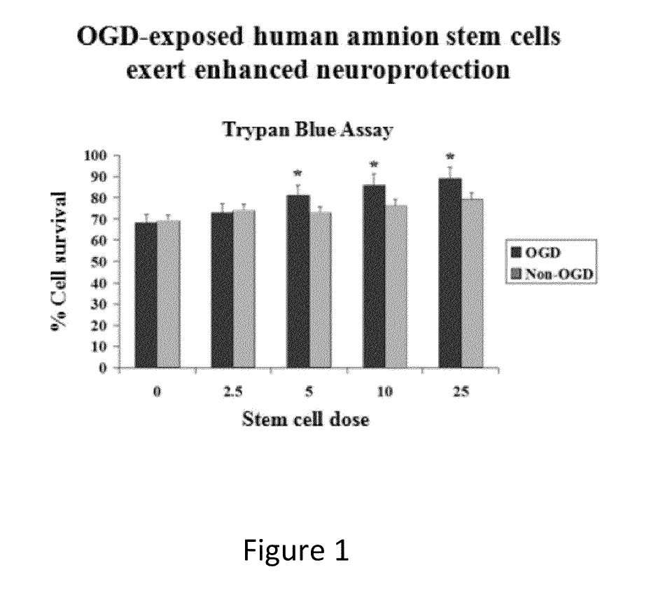

As demonstrated in FIGS. 1-3, in vitro experimental stroke (oxygen glucose deprivation, OGD) significantly increases levels of neurotrophic factors secreted by cultured human amnion stem cells compared to control, standard medium. The graph of FIG. 1 shows that OGD-exposed human amnion stem cells exert enhanced neuroprotection. As shown in FIG. 1, in vitro experimental stroke (oxygen glucose deprivation, OGD) significantly increases levels of neurotrophic factors secreted by cultured human amnion stem cells as compared to control, standard medium. Dose dependent increases in cell survival are shown.

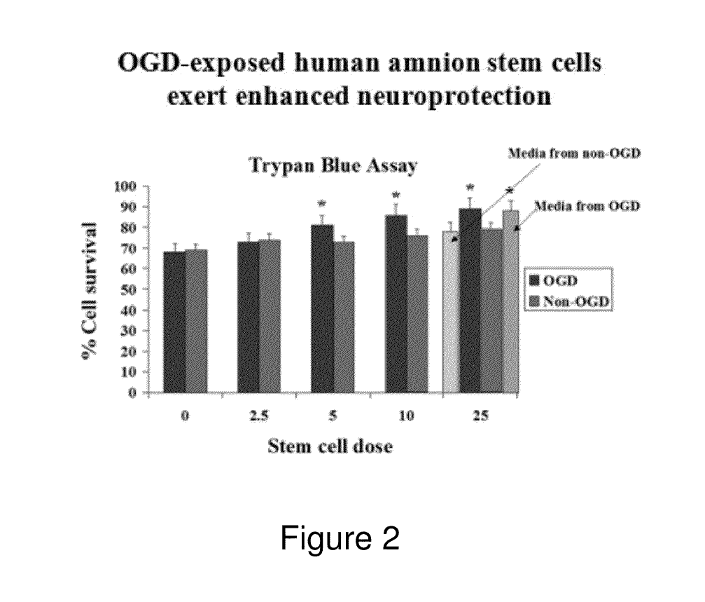

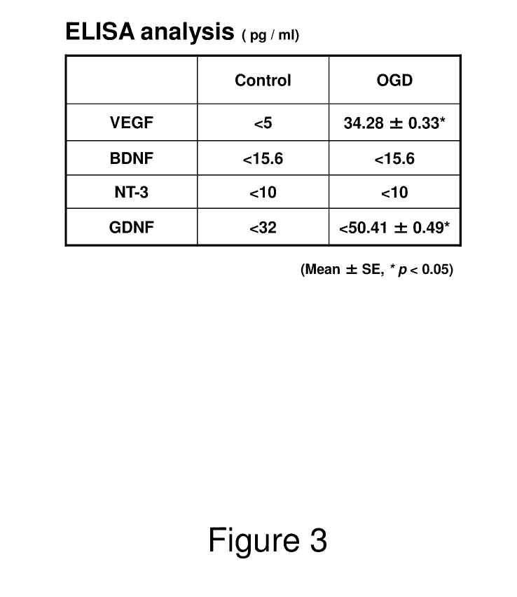

The graph of FIG. 2 illustrates that both OGD-exposed human amnion stem cells and the conditioned media from OGD-exposed amnion cells exert enhanced neuroprotection. As shown in FIG. 2, in vitro experimental stroke (oxygen glucose deprivation, OGD) significantly increases levels of neurotrophic factors secreted by cultured human amnion stem cells compared to control, standard medium. Surprisingly, the conditioned media from OGD-exposed amnion cells also exerts enhanced neuroprotection. As shown in FIG. 3, ELISA analysis results indicate that neurotrophic factors VEGF and GDNF are increased in OGD-exposed amnion cells.

### EXAMPLE 2

### Human Amniotic Epithelial Stem Cells Express Melatonin Receptor 1, But Not Melatonin Receptor 2

Recent studies have demonstrated that the human placenta is a good source of stem cells. The inventors have provided laboratory evidence that transplantation of these human placenta-derived cells in an in vivo stroke model promotes functional recovery. However, the mechanisms underlying these observed therapeutic benefits of human placenta-derived cells remain poorly understood. The inventors examined the expression of two discrete types of melatonin receptors and their role in proliferation and differentiation of cultured human amniotic epithelial cell (AECs).

Human AECs were obtained from the amnion, which was provided by Dr. Parolini under approved institutional guidelines. Immunocytochemical studies were performed to reveal: (1) melatonin receptor expression in cultured AECs, and: (2) proliferation and differentiation of cultured AECs with or without melatonin supplementation in the growth media.

AECs expressed melatonin receptor 1, but not melatonin receptor 2 as early as 3 days in vitro which peaked by 5 days in vitro. Furthermore, melatonin dose-dependently suppressed proliferation, but enhanced neural differentiation (TuJ1 and GFAP) of melatonin receptor 1-expressing AECs.

These results suggest a novel role for melatonin in modulating neural differentiation of human-placenta-derived AECs as donor cells for transplantation in neurological disorders. That melatonin receptor 1 rather than melatonin receptor 2 was detected in AECs, implicates melatonin receptor 1 as principally mediating these physiological effects of melatonin.

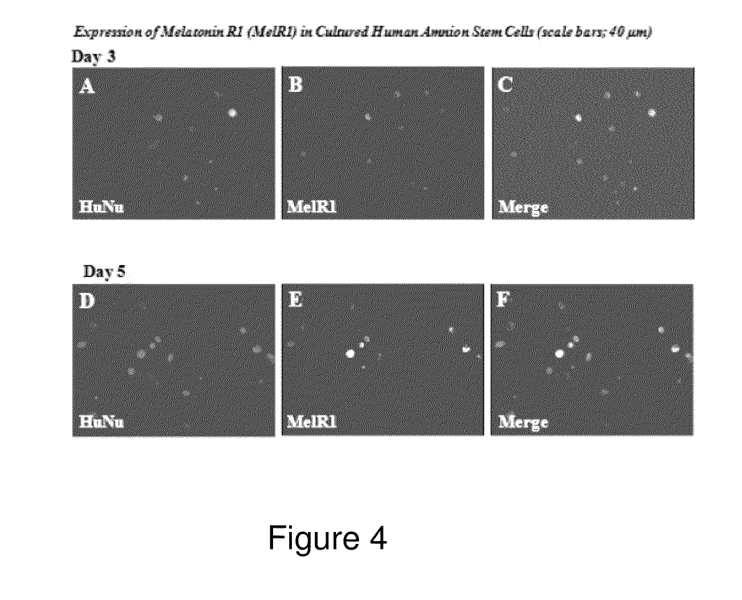

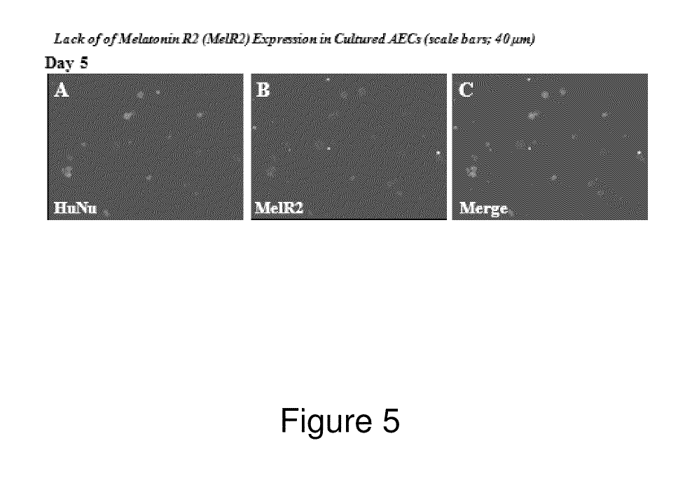

As demonstrated in FIGS. 4 and 5, human amnion stem cells expressed melatonin receptor 1 (MelR1), but not melatonin receptor 2 (MelR2). FIG. 4 shows the expression of Melatonin R1 (MelR1) in cultured human amnion stem cells after 3 and 5 days . FIGS. 4A-C show the expression of Human Specific Nuclear Antigen (HuNu) and Melatonin R1 receptor on day 3. The expression of HuNu indicates that the cells have differentiated into a neuronal phenotype. FIGS. 4D-F show the expression of human specific nuclear antigen (HuNu) and Melatonin R1 receptor on day 5. As can be seen in FIG. 5A-C, there is a lack of expression of the Melatonin Receptor 2 in cultured amnion epithelial stem cells.

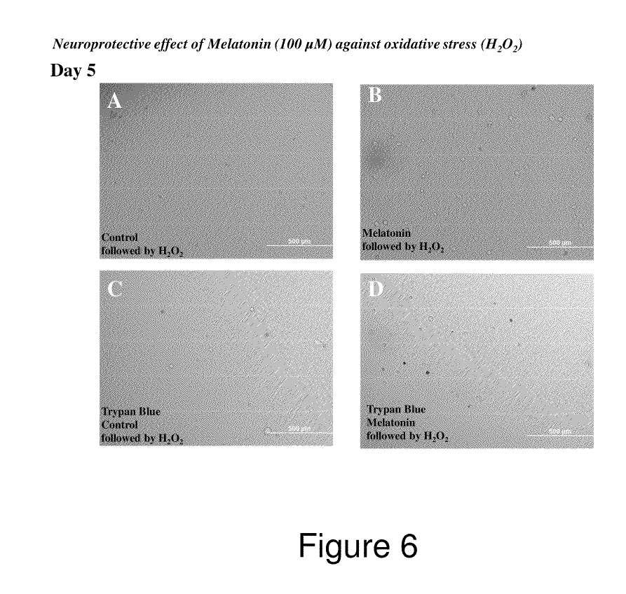

As demonstrated in FIGS. 6 through 8, in order to examine the neuroprotective effect of melatonin, cultured human amnion stem cells were exposed in the medium containing H202 at day 4. At day 5, cell survival was significantly reduced in non-melatonin treated cells. However, pre-treatment with melatonin protected against cell death. As shown in FIGS. 6A-D, cells that were pretreated with melatonin before undergoing oxidative stress, had a greater survival rate as compared to control samples which had undergone oxidative stress without pretreatment of the cells with melatonin. These results imply that melatonin exerts a neuroprotective effect on cells in a stroke model which can have implications in treating or preventing stroke in vivo.

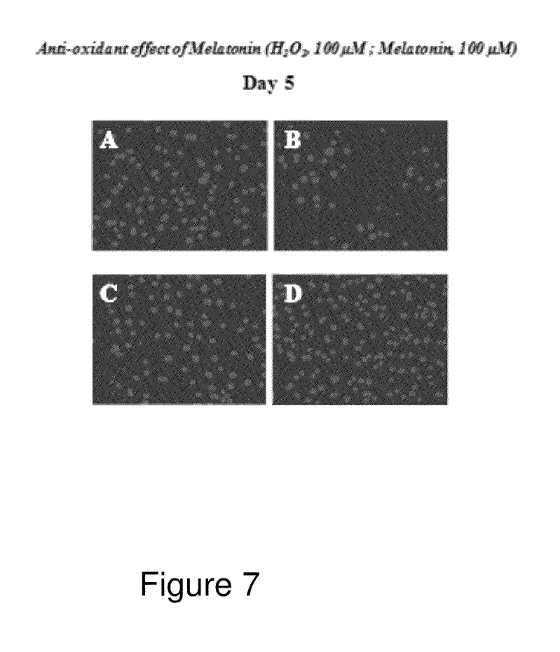

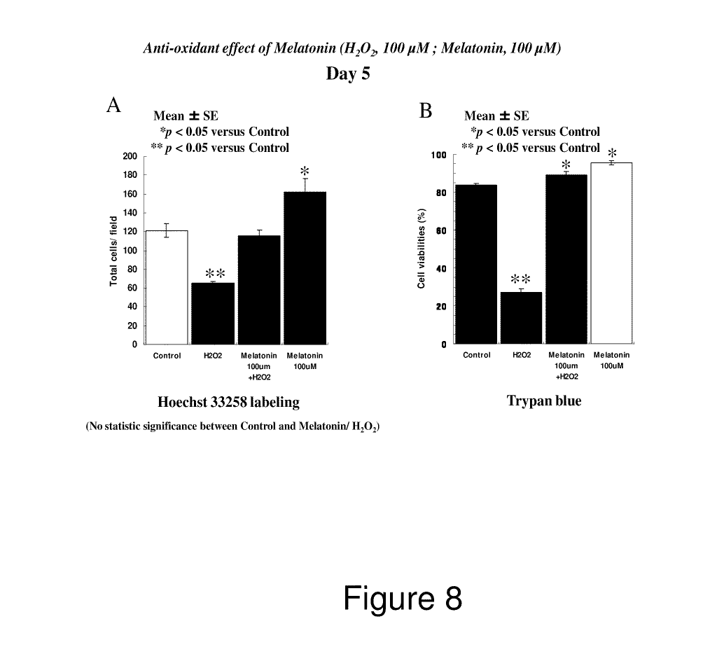

FIGS. 7A-D are images showing the anti-oxidant effect of melatonin on cells that are pretreated with 100 μM prior to undergoing oxidative stress. FIGS. 8A and 8B illustrate graphically that pretreatment of cells with melatonin enhances the neuroprotective effect on amnion epithelial cells. In cells treated with melatonin before oxidative stress is induced, the number of cells that survive are significantly greater than control cells that are exposed to oxidative stress.

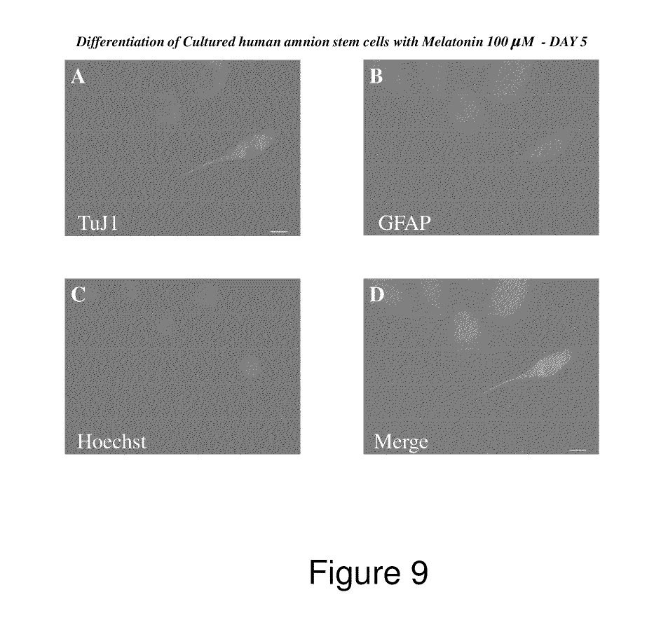

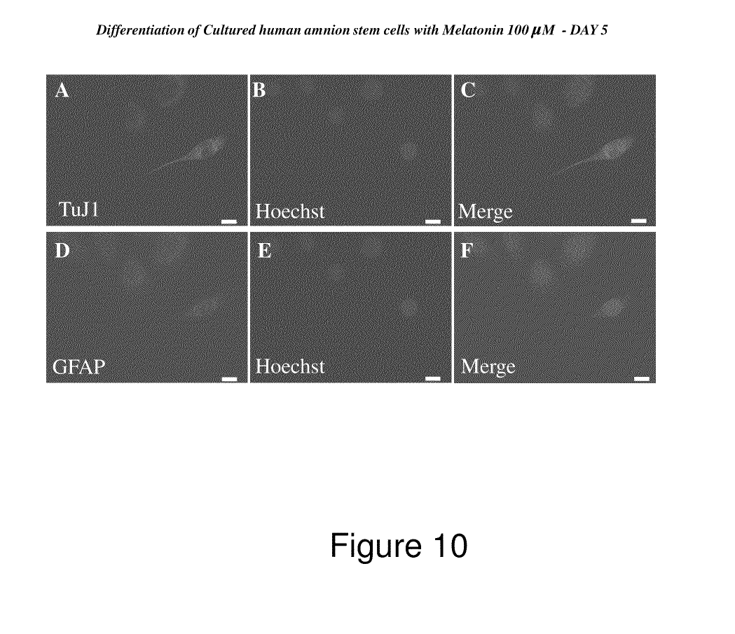

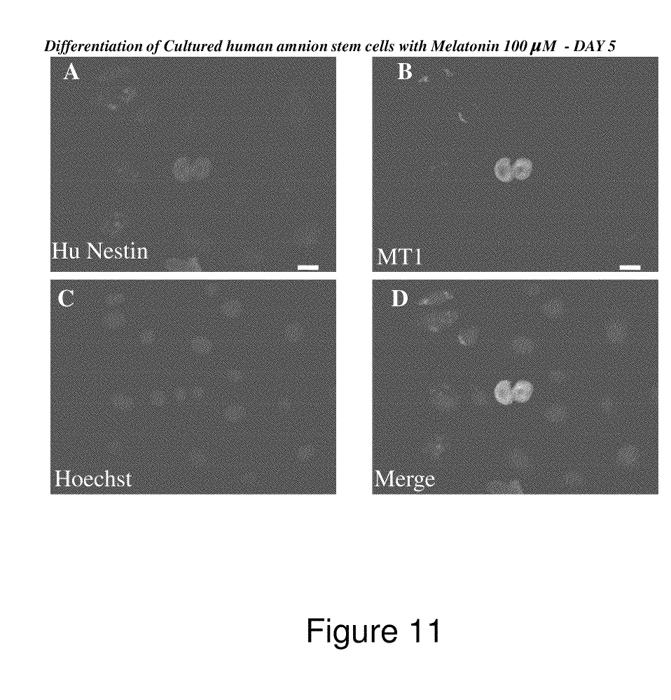

As demonstrated in FIGS. 9 through 14, melatonin enhanced human amnion stem cell differentiation into the neuronal cells as revealed by neuronal phenotype expression and neuron-like morphology. In addition, neuronal phenotype-expressing cells double-labeled with Melatonin receptor 1 further indicating the role of this particular receptor in human amnion stem cells differentiation. Expression of neuronal markers TuJ1 and GFAP are shown in FIGS. 9A-D and FIGS. 10A-F on day 5 following administration of melatonin. These results indicate differentiation of the amnion derived stem cells into neuronal cells after the administration of 100 μM Melatonin. Expression of neuronal markers HuNestin and MT1 are shown in FIGS. 11A-D on day 5 after administration of 100 μM melatonin to cultured amnion derived stem cells.

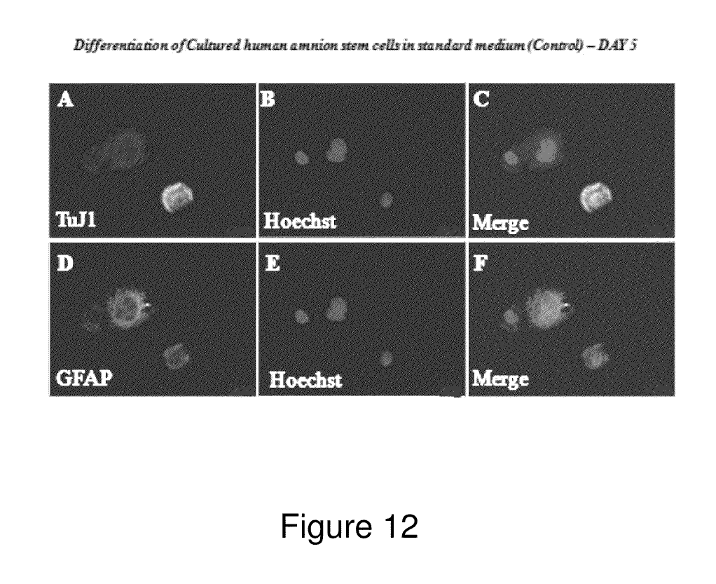

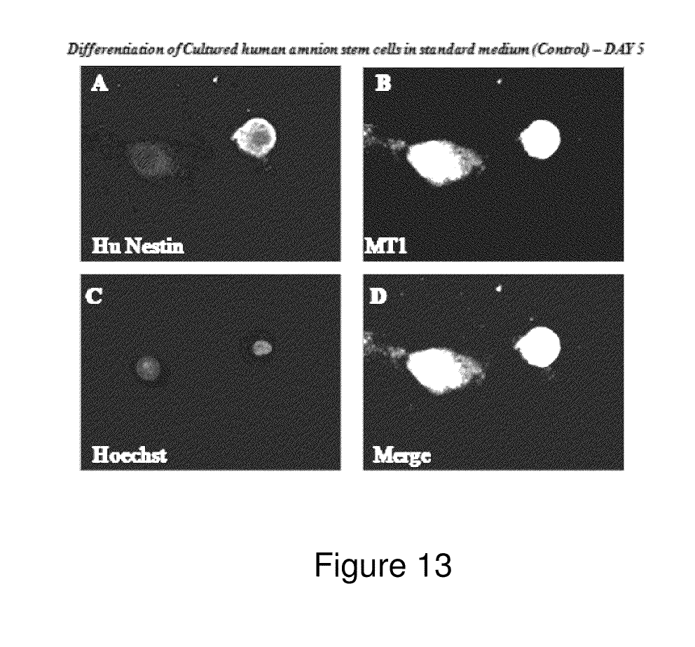

FIGS. 12A-F show the differentiation of cultured human amnion stem cells in standard medium (Control) on day 5 after administration of 100 μM Melatonin. Differentiation is shown by the expression of TuJ1 and GFAP. FIGS. 13A-D show differentiation of cultured human amnion stem cells in standard medium (Control) on day 5 after administration of 100 μM Melatonin. Differentiation is shown by expression of Hu Nestin and expression of MT1.

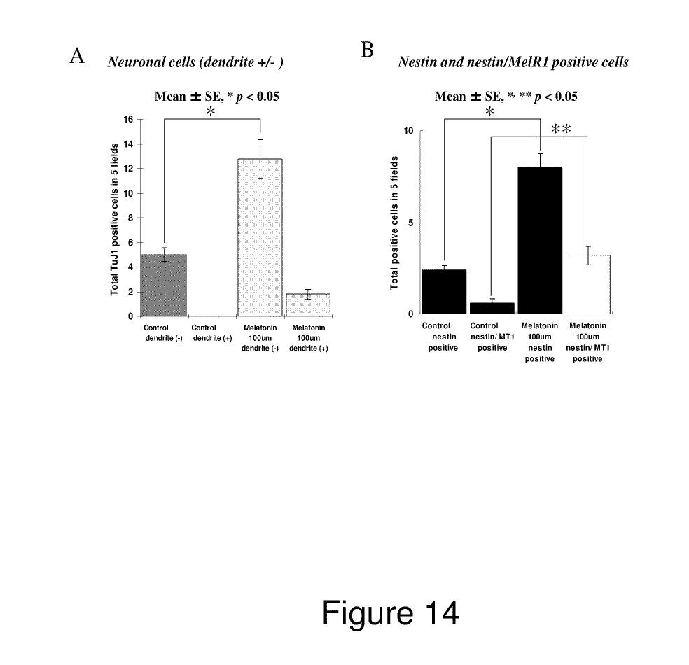

Melatonin was found to enhance human amnion stem cell differentiation into neuronal cells as revealed by neuronal phenotype expression and neuron-like morphology as shown in the graphs of FIG. 14A and 14B. In FIG. 14A, dendrite +/− neuronal cells that were either not treated with melatonin (control) or treated with 100 μM Melatonin were measured for Tull expression. The graph illustrates that the administration of melatonin increased the total number of cells expressing Tull, in comparison with control cells, regardless of whether the cells were dendrite positive or dendrite negative. The results show expression of TuJ1 is shown more in dendrite negative cells than dendrite positive cells. This is true for both control cells and cells treated with melatonin. The graph of FIG. 14B illustrates that the total number of positive cells in 5 fields for control or melatonin treated cells expressing nestin or nestin/MelR1. As shown, administration of 100 μM Melatonin increases the total number of nestin positive or nestin/MelR1 positive expressing cells as compared to controls.

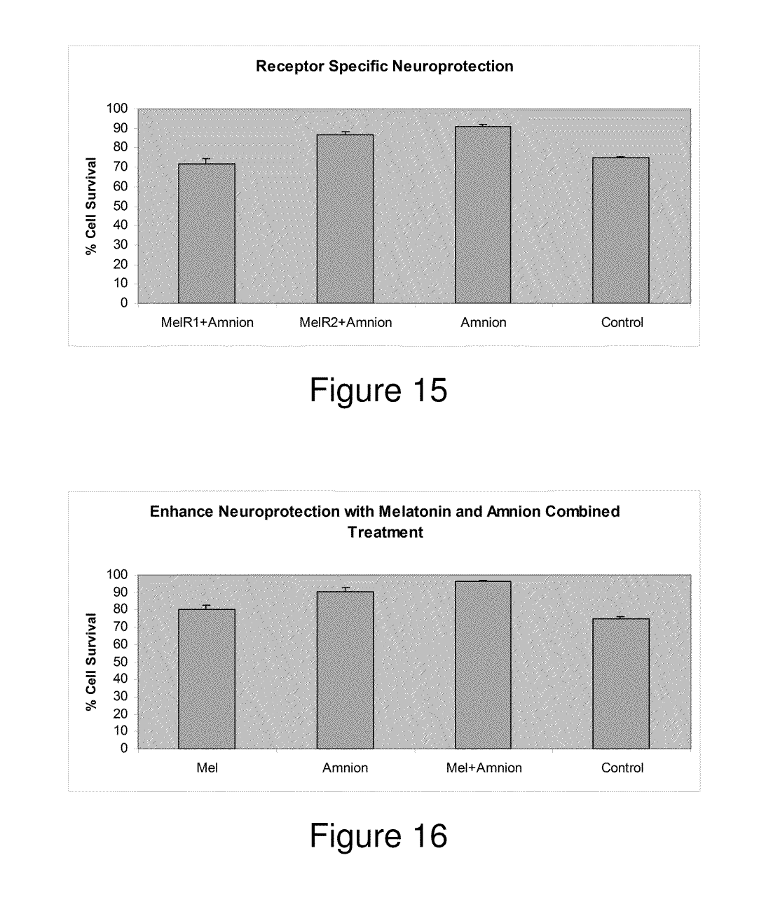

Pre-treatment of amnion cells with MelR1 antibody, but not MelR2 antibody, blocks neuroprotective effects of the stem cells on primary rat cells against in vitro experimental stroke as illustrated in FIG. 15. These results further support the claim that amnion-derived stem cells afford neuroprotection specifically via MelR1 receptor. To further support the claim that human amniotic epithelial cells (AECs) exert their neuroprotection via specific receptors, the inventors conducted receptor antibody blocking experiments in vitro. Pre-treatment of AECs with the melatonin receptor 1 (MelR1) antibody, but not the melatonin receptor 2 (MelR2) antibody blocks neuroprotective effects of the AECs on primary rat cells against the in vitro experimental stroke model of oxygen glucose deprivation (OGD). Cultured primary rat cells (gestation age 18; 20,0000 cells per well) were initially exposed to the OGD injury model (92% N2 and 8% 02 gas for 90 minutes). Cultured AECs (20,000 per well) grown in standard medium or in medium that was treated with either Me1R 1 or MelR2 (AECs incubated over 24 hours with either receptor antibody, with both antibodies prepared at 1 uM final concentrations) were subsequently added to the OGD-exposed cells. Based on the initial observations (see original data), a 25% AEC supplement to primary rat cell culture is therapeutically active, thus 5,000 AECs were added to 20,000 rat cells per culture well. Parallel sister OGD-exposed primary rat cells, without any co-culture with AECs, served as negative control. After 3 hours of co-culture treatment, cell viability (Trypan blue assay) was performed. Cell viability results revealed that OGD decreased primary rat cell survival (about 70% viability), whereas AEC co-culture treatment protected against such cell death (about 90% viability). These data replicated the original observations of AEC neuroprotection against experimental stroke. The inventors demonstrate that MelR1 antibody, but not MelR2 antibody pre-treatment blocked the neuroprotective effects of AECs, indicating that AEC-mediated therapeutic benefits are specifically regulated via MelR1 receptor.

Combined treatment with melatonin and amnion-derived stem cells enhanced the neuroprotective effects against in vitro experimental stroke as shown in FIG. 16. These data also lend support that stimulating the MelR1 could aid in the therapeutic benefits of amnion-derived stem cells. That MelR1 antibody regulated AEC neuroprotection suggests that stimulating this specific melatonin receptor is a potent target for enhancing therapeutic benefits. The inventors examined whether the combination of melatonin with AEC improved the neuroprotection against experimental stroke in vitro. The same primary rat cell culture and OGD paradigm as above was followed (i.e., 20,000 cells per well). Thereafter, AECs (5,000 per well), melatonin (100 uM), or AECs (2,500)+melatonin (50 uM) were added to the OGD-exposed cells. The inventors observed that while AECs and melatonin individually exerted neuroprotection, their combined treatment even at their sub-optimal levels afforded significantly improved protection against experimental stroke. These data have two-fold impact: 1) neuroprotection of AECs via the melatonin receptor is further indicated, and 2) a combination treatment whereby stimulating the melatonin receptor by the melatonin ligand in conjunction with AEC co-culture is robust therapeutic strategy for stroke treatment.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

In the preceding specification, all documents, acts, or information disclosed does not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually.

It is also to be understood that the following claims are intended to cover all of the generic and specific features of the invention herein described, and all statements of the scope of the invention, which, as a matter of language, might be said to fall there between. Now that the invention has been described,

What is claimed is:

1. A method of treating a patient suffering from a neurodegenerative disorder comprising administering a therapeutically effective amount of human placenta derived cells.

2. The method of claim 1 further comprising administering a therapeutically effective amount of melatonin.

3. The method of claim 1 wherein the placenta derived cells are exposed to a disease model in vitro prior to administration to a patient.

4. The method of claim 1 wherein the neurodegenerative disorder is selected from the group consisting of stroke, Alzheimer's disease, Parkinson's disease, and ischemia.

5. The method of claim 1 wherein the placenta derived cells are selected from the group consisting of amnion epithelial stem cells and amnion mesenchymal stem cells.

6. A method of regulating stem cells comprising stimulating the melatonin 1 receptor (MelR1).

7. The method of claim 6 further comprising administering a therapeutically effective dose of melatonin.

8. The method of claim 6 wherein the stem cells are human placenta derived stem cells.

9. The method of claim 8 wherein the stem cells are selected from the group consisting of amnion epithelial stem cells and amnion mesenchymal stem cells.

10. A method of enhancing neuroprotection in a patient comprising stimulating the melatonin receptor 1 (MelR1).

11. The method of claim 10 further comprising administering a therapeutically effective dose of melatonin and a therapeutically effective dose of human placenta derived stem cells.

12. The method of claim 11 wherein the human placenta derived stem cells are selected from the group consisting of amnion epithelial stem cells and amnion mesenchymal stem cells.
